(19) United States
(12) Patent Application Publication
(10) Pub. No.: US 2014/0364815 A1
(43) Pub. Date: Dec. 11, 2014

(54) Anti-flashback Needle

(71) Applicant: Philip Alexander Paspa, Hickory, NC (US)

(72) Inventor: Philip Alexander Paspa, Hickory, NC (US)

(21) Appl. No.: 14/300,953

(22) Filed: Jun. 10, 2014

Related U.S. Application Data
(60) Provisional application No. 61/956,476, filed on Jun. 10, 2013.

(51) Int. Cl. A61M 25/06

(52) U.S. Cl. CPC A61M 25/0693
USPC 604/272

(57) ABSTRACT

An anti-flashback needle inhibits the uncontrolled jetting of blood upon the insertion of the needle into a blood vessel such as an artery. The anti-flashback needle includes a shaft defining a lumen extending the length of the shaft; a hub connected to the shaft's proximal end, the hub having a hub wall defining a dissipation chamber in fluid communication with the lumen; and a flap connected to the hub and extending laterally across at least a portion of the dissipation chamber.

### CROSS-REFERENCE TO PRIORITY APPLICATION

This application hereby claims the priority benefit of pending U.S. Provisional Patent Application No. 61/956,476 for an “Anti-Flashback Needle” (filed Jun. 10, 2013 at the United States Patent and Trademark Office), which is hereby incorporated by reference in its entirety.

### FIELD OF THE INVENTION

The disclosure relates generally to medical equipment, and, more specifically, to anti-flashback needles.

### BACKGROUND OF THE INVENTION

During the course of medical procedures where percutaneous vascular access is contemplated, an arterial access needle is used. When a medical provider desires to position an object such as a stent into a blood vessel (e.g., vein, artery), the Seldinger technique may be employed. Using this technique, the medical provider inserts a guide wire (e.g., Seldinger wire) through the bore of the arterial access needle into the blood vessel. The guide wire may then be used to introduce the stent or other object into the blood vessel.

During insertion of the arterial access needle into the patient, the medical provider must ensure that the arterial access needle is in the blood vessel or there will not be proper placement of the guide wire. Typically, the medical provider detects that the needle is in close proximity to the blood vessel by detecting the pulsing blood as the needle rests outside the vessel wall. At that point, the medical provider advances the needle through the vessel wall. Typically, the medical provider confirms that the arterial access needle is in the proper position by observing the flashback (e.g., flash, backflow), which is the flow of blood out of the patient through the arterial flashback needle.

In the case of venous blood flow, the amount of flashback that is obtained from the arterial access needle is minimal as there is no pressure head. When an arterial access needle is placed into an artery, however, the pressure head is much greater, and the blood flows out of the patient with much greater force. Because this arterial flashback can be so forceful, it can result in a blood stream jetting a great distance from the arterial access needle. This jetting or pulsing effect increases the likelihood of blood hitting the medical professional or assistants, or of blood contaminating nearby surfaces or fomites.

There exists a need, therefore, for an arterial access needle that can reduce the likelihood of blood contamination of surrounding people and objects from flashback during the introduction of the arterial access needle into a blood vessel, particularly into an artery.

### SUMMARY OF THE INVENTION

The disclosure relates to an anti-flashback needle. In one aspect, the disclosure embraces an anti-flashback needle that includes a shaft defining a lumen extending the length of the shaft. The anti-flashback needle also includes a hub. The hub is connected to the shaft's proximal end. The hub has a hub wall that defines a dissipation chamber that is in fluid communication with the lumen. The anti-flashback needle also includes a flap. The flap is connected to the hub and extends laterally across at least a portion of the dissipation chamber.

In one embodiment, the flap is configured to allow the flap to be hingedly displaced about the flap's first end toward the distal portion of the hub, thereby accommodating the insertion of an object into the hub opening defined by the proximal portion of the hub and through the lumen.

In another embodiment, the flap extends laterally across between about forty percent and sixty percent of the diameter of the dissipation chamber.

In yet another embodiment, the flap is substantially comprised of rubber.

In yet another embodiment, the flap is substantially comprised of plastic.

In yet another embodiment, the flap has a living hinge to facilitate the flap's distal displacement about its first end.

In yet another embodiment, the anti-flashback needle includes an annular baffle positioned at the proximal portion of the dissipation chamber. The annular baffle is configured for disrupting the laminar flow of fluid streaming toward the proximal portion of the dissipation chamber.

In another aspect, the disclosure embraces an anti-flashback needle that includes a shaft defining a lumen extending the length of the shaft. The anti-flashback needle also includes a hub connected to the shaft's proximal end. The hub has a hub wall defining a dissipation chamber that is in fluid communication with the lumen. The anti-flashback needle also includes a disrupter. The disrupter is positioned within the dissipation chamber. The disrupter has an occluding member for partially obstructing the stream of fluid flowing through the dissipation chamber. The disrupter also has a connecting member for attaching the occluding member to the hub.

In one embodiment, the connecting member is substantially comprised of a resilient material that (i) allows the occluding member to be partially displaced by the force of a stream of fluid passing through the dissipation chamber and (ii) biases the occluding member in a position substantially along the dissipation chamber's longitudinal axis.

In another embodiment, the connecting member comprises a wire that is both flexible and resilient.

In another aspect, the anti-flashback needle comprises a shaft that defines a lumen extending the length of the shaft. The anti-flashback needle also includes a hub having a hub wall defining a dissipation chamber in fluid communication with the lumen. The distal portion of the hub wall is substantially undulating, thereby disrupting the laminar flow of fluid through the distal portion of the dissipation chamber.

In one embodiment, the distal portion of the hub wall undulates with a substantially fixed periodicity so as to define alternating wide and narrow portions of the distal portion of the dissipation chamber.

In another aspect, the anti-flashback needle includes a shaft and a hub. The hub is connected to the shaft's proximal end. The hub has a hub wall and a hub exo-wall. The hub wall defines a dissipation chamber in fluid communication with the lumen. The hub wall and the hub exo-wall define an exo-chamber in fluid communication with the dissipation chamber via an inner port defined by the hub wall. The anti-flashback needle also includes a laminar diverter positioned inside the dissipation chamber and substantially adjacent to the hub wall, the laminar diverter having a surface that is curved substantially toward the longitudinal axis of the hub. The laminar diverter is also positioned distally adjacent to the inner port, wherein, when a stream of fluid is introduced into the distal end of the dissipation chamber, the laminar diverter substantially redirects the flow of at least a portion of the fluid by the Coanda effect out of the dissipation chamber and into the exo-chamber via the first opening. The hub exo-wall defines an outer port through which fluid may exit the exo-chamber.

In one embodiment, the laminar diverter is integral with the hub wall.

In another embodiment, the inner port is offset from the outer port, thereby facilitating the exo-chamber's functionality as a baffle for dissipating the force of fluid exiting the anti-flashback needle via the outer port.

In another aspect, the disclosure embraces an anti-flashback needle that includes a shaft, a hub, and a vane. The vane is attached to the hub wall and extends into the distal portion of the dissipation chamber, thereby disrupting the laminar flow of fluid entering the dissipation chamber from the lumen.

The foregoing, as well as other objectives and advantages of the disclosure, and the manner in which the same are accomplished, are further specified within the following detailed description and its accompanying drawings.

### BRIEF DESCRIPTION OF THE DRAWINGS

FIG. 1 is a semi-transparent side perspective view of an exemplary anti-flashback needle having a flap according to the present disclosure.

FIG. 2 is a side cross-sectional view of an exemplary anti-flashback needle having a flap according to the present disclosure.

FIG. 3 is a semi-transparent side perspective view of an alternative embodiment of an exemplary anti-flashback needle having a disrupter according to the present disclosure.

FIG. 4 is a side cross-sectional view of an alternative embodiment of an exemplary anti-flashback needle having a disrupter according to the present disclosure.

FIG. 5 is a semi-transparent side perspective view of an alternative embodiment of an exemplary anti-flashback needle having an undulating hub wall according to the present disclosure.

FIG. 6 is a side cross-sectional view of an alternative embodiment of an exemplary anti-flashback needle having an undulating hub wall according to the present disclosure.

FIG. 7 is a semi-transparent side perspective view of an alternative embodiment of an exemplary anti-flashback needle having a laminar diverter according to the present disclosure.

FIG. 8 is a side cross-sectional view of an alternative embodiment of an exemplary anti-flashback needle having a laminar diverter according to the present disclosure.

FIG. 9 is a semi-transparent side perspective view of an alternative embodiment of an exemplary anti-flashback needle having a vane according to the present disclosure.

FIG. 10 is a side cross-sectional view of an alternative embodiment of an exemplary anti-flashback needle having a vane according to the present disclosure.

### DETAILED DESCRIPTION

The disclosure relates to an anti-flashback needle. In various exemplary embodiments, the anti-flashback needle is configured to disrupt the flow of fluid (e.g., blood, bodily fluids, etc.) moving from the anti-flashback needle's distal end to the anti-flashback needle's proximal end. Typically, the fluid stream flowing through the anti-flashback needle is under significant pressure from a pressure head. Typically, the fluid stream is blood flow emanating from a blood vessel. More typically, the blood flow emanates from an artery (e.g., a human artery) because arterial blood flow is under much greater pressure than venous blood flow. When the anti-flashback needle is inserted into the artery, the arterial blood pressure causes the blood to spurt (e.g., jet, pulse) through the shaft and out through the hub. Unless measures are taken to dissipate the force of this jetting blood, the blood can spurt onto nearby people or objects.

In various exemplary embodiments, the anti-flashback needle according to the present disclosure adopts various configurations to inhibit the laminar flow of blood through the anti-flashback needle. In other words, the various configurations of the anti-flashback needle adapter are intended to convert the stream of blood from a substantially laminar flow to a substantially turbulent flow. The greater the turbulence that can be created, the greater reduction in velocity of the blood flow. As the velocity is reduced, the jetting of blood can be likewise reduced, ideally to a trickle that stays below the fingertips of the user (e.g., medical professional) of the anti-flashback needle adapter. By reducing the velocity of the stream of blood exiting the anti-flashback needle adapter according to the present invention, the likelihood of contamination of nearby persons and/or objects is greatly reduced.

In this disclosure, a convention is followed wherein the term “proximal” refers to the portion of the device closest to the user (e.g., medical provider, medical professional, practitioner) when the device is being percutaneously inserted into a patient per its intended use (e.g., for arterial catheterization purposes). Furthermore, the term distal refers to the portion of the device nearest the patient (e.g., farthest from the medical provider) when the device is being percutaneously inserted into a patient in the proper way.

Furthermore, references to arterial access by the anti-flashback needle according to the present disclosure are for purposes of illustrating an intended use. These illustrative references are not intended to limit the disclosure in any way. To the contrary, the anti-flashback needle according to the present disclosure may be useful for providing access to other blood vessels or anatomical structures. Moreover, aspects of the present disclosure may be useful for gaining access to any enclosed space filled with pressurized fluid.

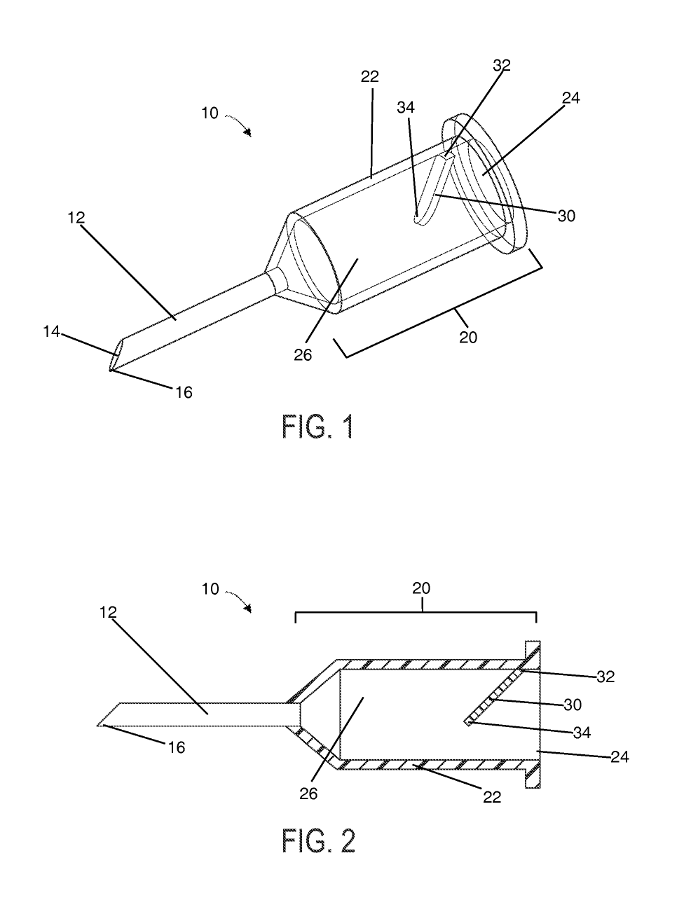

Referring now to FIGS. 1 and 2, an exemplary embodiment of the anti-flashback needle 10 according to the present disclosure includes a shaft 12 defining a lumen 14 (e.g., bore) extending the length of the shaft 12. Typically, the distal end of the shaft 12 includes a bevel 16 which facilitates piercing of tissue such that the anti-flashback needle 10 can be more easily advanced into a subcutaneous position and can more easily penetrate arterial walls. The lumen 14 provides an avenue for transmitting fluid from the shaft's distal end to the shaft's proximal end. Typically, the shaft 12 is constructed of a rigid material (e.g., stainless steel) and may be of a variety of gauges and lengths depending upon the intended application.

The anti-flashback needle 10 according to the present disclosure also includes a hub 20. The hub 20 is connected to the proximal end of the shaft 12. Typically, the hub 20 is substantially cylindrical, having a hub wall 22 that defines a dissipation chamber 26 positioned inside the hub 20. Typically, the dissipation chamber 26 is also substantially cylindrical and is in fluid communication with the lumen 14.

The hub wall 22 also defines a hub opening 24 at the proximal end of the hub 20. The hub opening 24 provides access to the anti-flashback needle 10 from the proximal end. Typically, the hub opening 24 is utilized by the medical professional for intra-arterial insertion of objects (e.g., catheter guide wire) through the anti-flashback needle 10. Typically, the hub 20 is fixably attached to the shaft 12, though in some embodiments the hub 20 and shaft 12 may be integral. Typically, the hub 20 is constructed of plastic. Furthermore, the hub 20 may be configured for attaching to another device or instrument (e.g., a needle adapter, syringe). In one embodiment, the hub 20 is configured for attaching to another device or instrument using a Luer lock system.

Typically, the dissipation chamber 26 is configured for dissipating the force of the fluid stream passing into the dissipation chamber 26 from the lumen 14. The exemplary anti-flashback needle 10 illustrated in FIGS. 1 and 2 includes a flap 30 connected to the hub 20 and extending laterally across at least a portion of the dissipation chamber 26. The flap 30 is positioned such that the fluid stream passing through the dissipation chamber 26 will contact the flap 30, and the resistance provided by the flap 30 will disrupt the laminar flow of fluid, create turbulence, and consequently reduce the force of the fluid stream.

Because the flap 30 must be positioned to intercept the fluid jetting from the proximal end of the lumen 14, the flap 30 typically extends into the longitudinal axis portion of the hub 20. Typically, the flap 30 extends laterally across between about forty percent and sixty percent (e.g., about fifty percent) of the diameter of the dissipation chamber 26. Because objects are typically inserted into the hub opening 24 substantially along the hub's longitudinal axis, the flap 30 is configured so that it does not impede the insertion of such objects. More particularly, the flap 30 is configured to allow the flap 30 to be hingedly displaced about the flap's first end toward the distal portion of the hub 20, thereby accommodating the insertion of an object into the hub opening 24 and through the lumen 14. In other words, when a rigid or semi-rigid object is passed through the hub 20, the object distally displaces the flap 30 thereby allowing the object to pass through the dissipation chamber 26 and into the lumen 14.

Typically, the flap 30 is substantially comprised of a resilient material (e.g. rubber) that allows the flap 30 to be distally displaced and then return to its neutral position extending into the dissipation chamber 26. In an alternative embodiment, the flap 30 may be substantially comprised of plastic. The distal displacement of the plastic flap may be facilitated by incorporating a living hinge into the flap. A living hinge may also be useful in facilitating the displacement of flaps created from other materials.

In one embodiment, the anti-flashback needle 10 may also include an annular baffle 36. Typically, the annular baffle 36 is positioned at the proximal portion of the dissipation chamber 26. The annular baffle 36 is configured for disrupting the laminar flow of fluid streaming toward the proximal portion of the dissipation chamber 26. The annular baffle 36 is typically integral with the hub wall 22 and extends substantially around the entire interior circumference of the proximal portion of the hub 20.

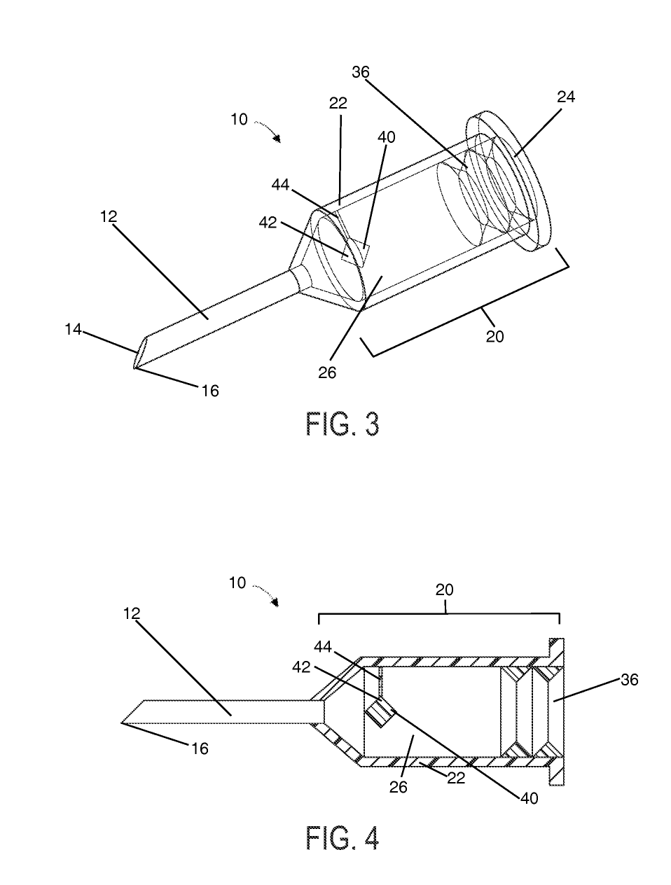

An alternative embodiment of the present disclosure depicted in FIGS. 3 and 4 embraces an anti-flashback needle 10 having a shaft 12 defining a lumen 14, and a hub 20 having a hub wall 22 defining a dissipation chamber 26. This alternative embodiment includes a disrupter 40 positioned within the dissipation chamber 26. The disrupter 40 includes an occluding member 42 for partially obstructing the stream of fluid flowing through the dissipation chamber 26. The disrupter 40 also includes a connecting member 44 for attaching the occluding member 42 to the hub 20.

The disrupter 40 effectively reduces the kinetic energy of the fluid stream by forcing the fluid ejected from the lumen 14 around the occluding member 42. The occluding member 42 may be shaped in such a way as to promote turbulence in the fluid stream, thereby dissipating its energy.

The connecting member is typically constructed substantially from a resilient material that (i) allows the occluding member to be partially displaced by the force of a stream of fluid passing through the dissipation chamber and (ii) biases the occluding member in a position substantially along the dissipation chamber's longitudinal axis. The use of a resilient material advantageously allows the occluding member 42 to be distally displaced, thereby facilitating the insertion of a rigid or semi-rigid object through the hub 20 and lumen 14 and into the artery. The connecting member 44 may include a wire that is both flexible and resilient.

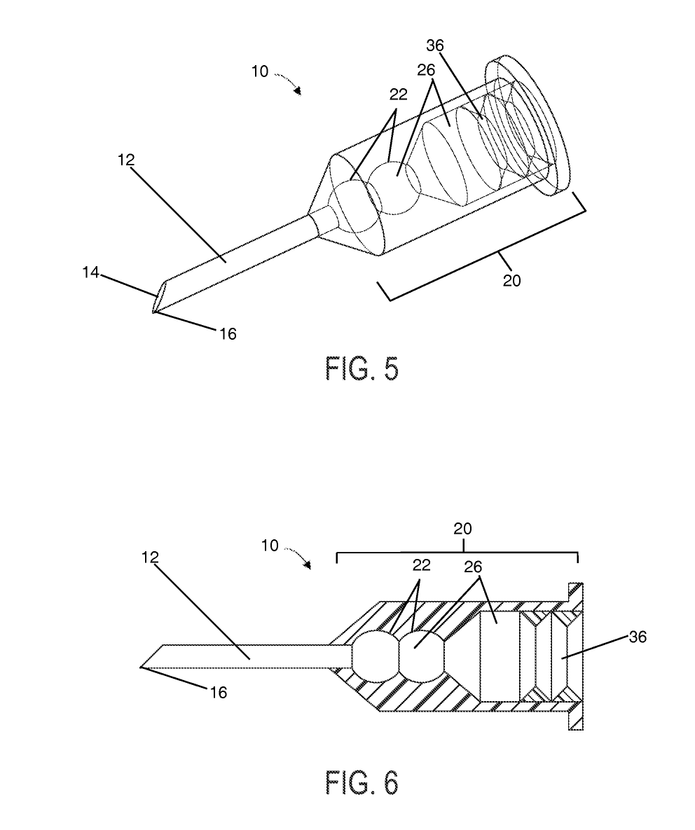

Referring now to FIGS. 5 and 6, an alternative embodiment of the present disclosure embraces an anti-flashback needle 10 having a shaft 12 defining a lumen 14, and a hub 20 having a hub wall 22. The hub wall 22 defines a dissipation chamber 26 in fluid communication with the lumen 14. In this alternative embodiment, the distal portion of the hub wall 22 is substantially undulating. In other words, the distal portion of the dissipation chamber 26 does not take a cylindrical shape. Instead, the undulations of the distal portion of the hub wall 22 cause the distal portion of the dissipation chamber 26 to vary in diameter from point to point along the longitudinal axis of the hub 20. The undulations in the hub wall 22 disrupt the laminar flow of fluid as it pass through the distal portion of the dissipation chamber 26. The generation of turbulence in the fluid stream dissipates the force of the fluid stream, thereby reducing the risk of undesired jetting during flashback.

In an alternative embodiment, the distal portion of the hub wall 22 undulates with a substantially fixed periodicity so as to define alternating wide and narrow portions of the distal portion of the dissipation chamber.

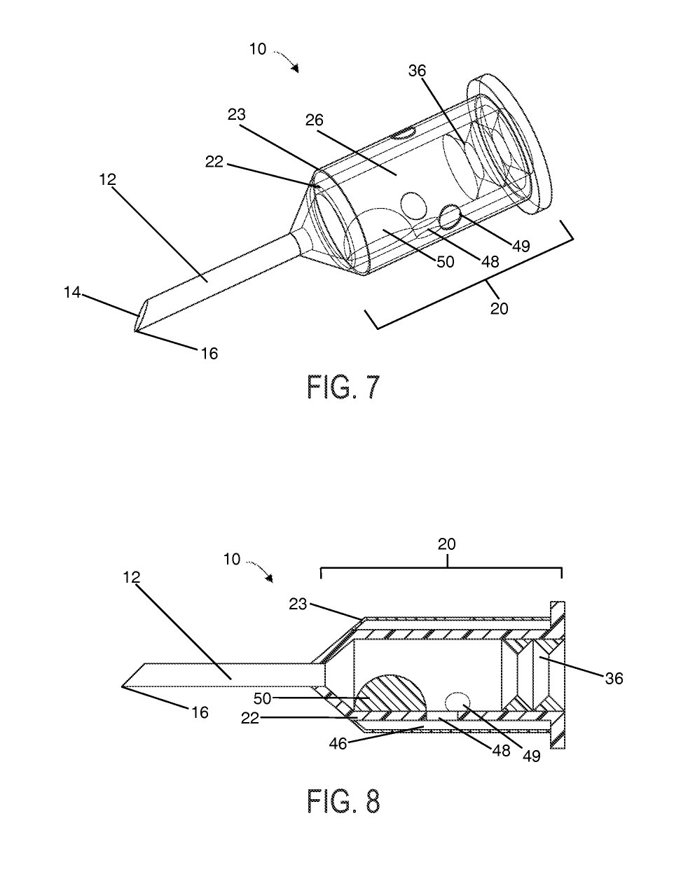

Turning now to FIGS. 7 and 8, an alternative embodiment of the present disclosure embraces an anti-flashback needle that includes a hub 20 having a hub wall 22 and a hub exo-wall 23. The hub exo-wall 23 typically forms a cylinder that shares the same longitudinal axis as the cylinder formed by the hub wall 22, with the cylinder formed by the hub exo-wall having a greater diameter than the cylinder formed by the hub wall 22. The hub wall 22 and hub exo-wall 23 define an exo-chamber 46. The exo-chamber 46 is in fluid communication with the dissipation chamber 26. The hub wall 22 defines an inner port (e.g., aperture) which allows fluid to move from the dissipation chamber 26 to the exo-chamber 46. When fluid, such as blood, enters the exo-chamber 46, it can exit the anti-flashback needle 10 through an outer port defined by the hub exo-wall 23.

When fluid enters the dissipation chamber 26, it tends to stream along the same axis as the lumen 14. If the stream were allowed to continue on this path unabated, it would exit the hub opening 24 with a substantial amount of force, potentially resulting in undesired jetting. Therefore, the alternative embodiment of the anti-flashback needle 10 depicted in FIGS. 5 and 6 incorporates a structure for diverting at least a portion of the fluid through the inner port 48 and into the exo-chamber 46. More particularly, a laminar diverter 50 is positioned inside the dissipation chamber 26 and substantially adjacent to the hub wall 22. Typically, the laminar diverter 50 has a surface that is substantially curved toward the longitudinal axis of the hub 20. In other words, the laminar diverter 50 forms a substantially spherical surface that protrudes from the hub wall 22 into the dissipation chamber 26. Typically, the laminar diverter 50 is integral with the hub wall 20, thereby forming a mounded segment of the hub wall 20.

The laminar diverter 50 is also positioned distally adjacent to the inner port, thereby facilitating the diversion of the fluid stream toward the inner port 48. The laminar diverter's curved shape utilizes the Coanda effect to substantially redirect the flow of at least a portion of the fluid out of the dissipation chamber 26 and into the exo-chamber via the inner port 48. In other words, as the fluid stream passes near the laminar diverter 50, the fluid stream tends to follow the curvature of the laminar diverter 50 by virtue of the Coanda effect. The inner port 48 is advantageously positioned adjacent to the proximal portion of the laminar diverter 50 in a general position to intercept the redirected flow of fluid.

As mentioned, as the fluid passes through the inner port 48 it enters the exo-chamber 46. Although the laminar diverter 50 has redirected the fluid stream, the fluid stream may still have enough velocity as it pass through the inner port 48 to result in jetting if it were to immediately exit the anti-flashback needle 10. To reduce this possibility, the inner port 48 is offset from the outer port 49 (e.g., their center points are not on the same axis). More typically, the inner port 48 is at least 90 degrees from the outer port 49, thereby causing the fluid stream to travel circumferentially around the exo-chamber 46 before the fluid stream can exit the anti-flashback needle 10 through the outer port 49. In this way, the exo-chamber operates as an outflow baffle reducing the velocity of the fluid stream exiting the anti-flashback needle. Typically, this baffling will result in the fluid stream existing the anti-flashback needle as a trickle instead of a jet or spurt.

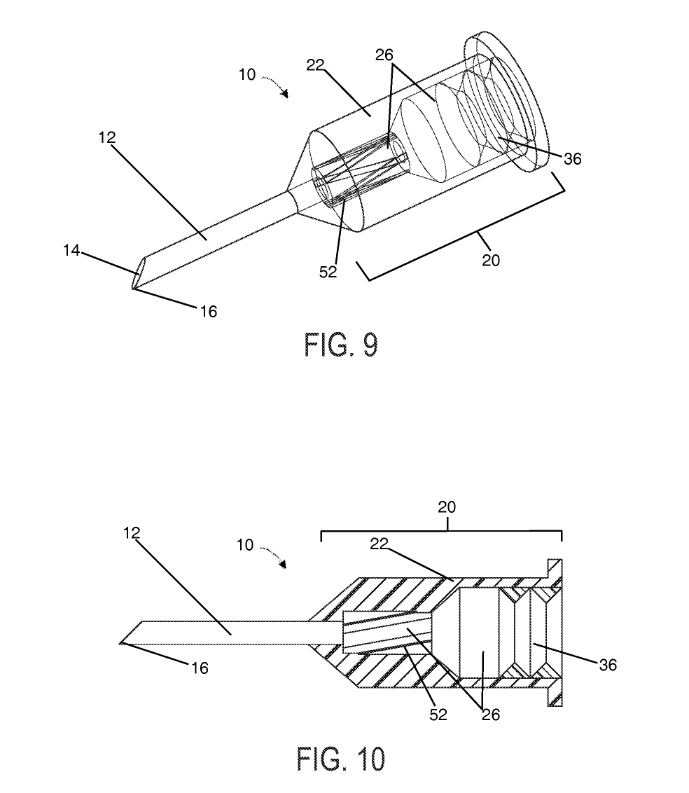

Reference is now made to FIGS. 9 and 10. In another alternative embodiment, the present disclosure embraces an anti-flashback needle 10 having a shaft 12 defining a lumen 14 extending the length of the shaft, a hub 20 having a hub wall 22 defining a dissipation chamber 26 in fluid communication with the lumen 14, and vane attached to the hub wall. The vane extends from the hub wall 22 toward the longitudinal axis of the hub 20. The vane is positioned in the distal portion of the dissipation chamber 26 such that as the fluid stream exits the lumen 14 it contacts the vane 52. More typically, a plurality of vanes 52 are positioned within the distal portion of the dissipation chamber 26. The vanes may be angled or spiral. As the fluid stream contacts the vanes 52, the laminar flow of the fluid stream is disrupted (e.g., turbulence is introduced). The force of the fluid stream is thereby diminished, thus reducing the possibility of unwanted jetting of fluid onto surrounding objects or people as the fluid exits the anti-flashback needle.

In the specification and figures, typical embodiments of the invention have been disclosed. The present invention is not limited to such exemplary embodiments. Unless otherwise noted, specific terms have been used in a generic and descriptive sense and not for purposes of limitation.

1. An anti-flashback needle, comprising:
a shaft defining a lumen extending the length of the shaft;
a hub connected to the shaft's proximal end, the hub having a hub wall defining a dissipation chamber in fluid communication with the lumen;
a flap connected to the hub and extending laterally across at least a portion of the dissipation chamber.

2. The anti-flashback needle of claim 1, wherein the flap is configured to allow the flap to be hingedly displaced about the flap's first end toward the distal portion of the hub, thereby accommodating the insertion of an object into the hub opening defined by the proximal portion of the hub and through the lumen.

3. The anti-flashback needle of claim 1, wherein the flap extends laterally across between about forty percent and sixty percent of the diameter of the dissipation chamber.

4. The anti-flashback needle of claim 1, wherein the flap is substantially comprised of rubber.

5. The anti-flashback needle of claim 1, wherein the flap is substantially comprised of plastic.

6. The anti-flashback needle of claim 1, wherein the flap has a living hinge to facilitate the flap's distal displacement.

7. The anti-flashback needle of claim 1, comprising:
an annular baffle positioned at the proximal portion of the dissipation chamber, wherein the annular baffle is configured for disrupting the laminar flow of fluid streaming toward the proximal portion of the dissipation chamber.

8. An anti-flashback needle, comprising:
a shaft defining a lumen extending the length of the shaft;
a hub connected to the shaft's proximal end, the hub having a hub wall defining a dissipation chamber in fluid communication with the lumen;
a disrupter positioned within the dissipation chamber, the diverter having (i) an occluding member for partially obstructing the stream of fluid flowing through the dissipation chamber and (ii) a connecting member for attaching the occluding member to the hub.

9. The anti-flashback needle of claim 8, wherein the connecting member is substantially comprised of a resilient material that (i) allows the occluding member to be partially displaced by the force of a stream of fluid passing through the dissipation chamber and (ii) biases the occluding member in a position substantially along the dissipation chamber's longitudinal axis.

10. The anti-flashback needle of claim 8, wherein the connecting member comprises a wire that is both flexible and resilient.

11. The anti-flashback needle of claim 8, comprising:
an annular baffle positioned at the proximal portion of the dissipation chamber, wherein the annular baffle is configured for disrupting the laminar flow of fluid streaming toward the proximal portion of the dissipation chamber.

12. An anti-flashback needle, comprising:
a shaft defining a lumen extending the length of the shaft; and
a hub connected to the shaft's proximal end, the hub having a hub wall defining a dissipation chamber in fluid communication with the lumen;
wherein the distal portion of the hub wall is substantially undulating, thereby disrupting the laminar flow of fluid through the distal portion of the dissipation chamber.

13. The anti-flashback needle of claim 12, wherein the distal portion of the hub wall undulates with a substantially fixed periodicity so as to define alternating wide and narrow portions of the distal portion of the dissipation chamber.

14. The anti-flashback needle of claim 12, comprising:
an annular baffle positioned at the proximal portion of the dissipation chamber, wherein the annular baffle is configured for disrupting the laminar flow of fluid streaming toward the proximal portion of the dissipation chamber.

15. An anti-flashback needle, comprising:
a shaft defining a lumen extending the length of the shaft; and
a hub connected to the shaft's proximal end; the hub having a hub wall and a hub exo-wall, wherein the hub wall defines a dissipation chamber in fluid communication with the lumen and wherein the hub wall and hub exo-wall define an exo-chamber in fluid communication with the dissipation chamber via a inner port defined by the hub wall;
a laminar diverter positioned (i) inside the dissipation chamber and substantially adjacent to the hub wall, the laminar diverter having a surface that is curved substantially toward the longitudinal axis of the hub and (ii) distally adjacent to the first opening, wherein, when a stream of fluid is introduced into the distal end of the dissipation chamber, the laminar diverter substantially redirects the flow of at least a portion of the fluid by the Coanda effect out of the dissipation chamber and into the exo-chamber via the first opening;
wherein the hub exo-wall defines a outer port through which fluid may exit the exo-chamber.

16. The anti-flashback needle of claim 15, wherein the laminar diverter is integral with the hub wall.

17. The anti-flashback needle of claim 15, wherein the inner port is offset from the outer port, thereby facilitating the exo-chamber's functionality as a baffle for dissipating the force of fluid exiting the anti-flashback needle via the outer port.

18. The anti-flashback needle of claim 15, comprising:
an annular baffle positioned at the proximal portion of the dissipation chamber, wherein the annular baffle is configured for disrupting the laminar flow of fluid streaming toward the proximal portion of the dissipation chamber.

19. An anti-flashback needle, comprising:
a shaft defining a lumen extending the length of the shaft;
a hub connected to the shaft's proximal end, the hub having a hub wall defining a dissipation chamber in fluid communication with the lumen; and
a vane attached to the hub wall and extending into the distal portion of the dissipation chamber, thereby disrupting the laminar flow of fluid entering the dissipation chamber from the lumen.

20. The anti-flashback needle of claim 19, comprising:
an annular baffle positioned at the proximal portion of the dissipation chamber, wherein the annular baffle is configured for disrupting the laminar flow of fluid streaming toward the proximal portion of the dissipation chamber.
